(19) United States
(12) Patent Application Publication
(10) Pub. No.: US 2021/0115381 A1
(43) Pub. Date: Apr. 22, 2021

(54) THERMOFLUIDICS FOR SPATIAL CONTROL OF GENE ACTIVATION

(71) Applicants: UNIVERSITY OF WASHINGTON, Seattle, WA (US); WILLIAM MARSH RICE UNIVERSITY, Houston, TX (US)

(72) Inventors: Kelly Rose Stevens, Seattle, WA (US); Daniel Corbett, Seattle, WA (US); Jordan Miller, Houston, TX (US); Bagrat Grigoryan, Houston, TX (US)

(21) Appl. No.: 17/073,165

(22) Filed: Oct. 16, 2020

Related U.S. Application Data
(60) Provisional application No. 62/916,620, filed on Oct. 17, 2019.

(51) Int. Cl. C12M 1/02; C12M 1/00; C12M 1/12

(52) U.S. Cl. CPC C12M 41/18; C12M 25/14; C12M 45/20

(57) ABSTRACT

The disclosure provides biocompatible heat exchangers, artificial tissues, systems, and thermofluidic methods for spatiotemporal control of biological signaling and gene expression. The disclosure demonstrates that in heat exchangers containing embedded cells with heat-activatable transgenes, gene expression patterning can be tuned both spatially and dynamically by varying channel network architecture, fluid temperature, fluid flow direction, and stimulation timing in a user-defined manner and maintained in vivo.

### CROSS-REFERENCE TO RELATED APPLICATION

This application claims the benefit of U.S. Provisional Application 62/916,620, filed Oct. 17, 2019, the disclosure of which is incorporated herein by reference in its entirety.

### STATEMENT OF GOVERNMENT LICENSE RIGHTS

This invention was made with government support under Grant No. HL137188, awarded by the National Institutes of Health. The government has certain rights in the invention.

### BACKGROUND

Human development, homeostasis, regeneration, and pathology are broadly controlled by activation of genes, which elicit downstream cellular responses. Such gene activation is often defined both spatially and temporally. However, systems that allow users to induce gene expression typically lack refined spatial and temporal control. Technologies that enable users to reliably control where, when and how much a specific gene is expressed are highly needed and have the potential for tremendous value as a tool in basic science and medical practice.

For example, the ability to spatially activate genes in bioengineered tissues could be used to drive emergent cell fate and assembly decisions, similar to those found in response to morphogen gradients in development. It could further be used in the context of an inducible expression tumor model to spatially map the genes responsible for epithelial-mesenchymal transition, a cellular response that plays role in turning a tumor from dormant to metastatic. Moreover, an artificial tissue can be engineered to controllably activate a therapeutic gene after implantation, such as an artificial pancreas that can be induced to produce insulin in diabetic patients.

To control gene expression, biologists have developed diverse technologies to rewire cells at the genetic level, such as gene knockout, inhibition, overexpression, and editing. To further enable spatial and dynamic control of gene expression, several of these tools have been adapted to be triggered by exogenous stimuli such as light (e.g., optogenetic transcriptional control). Light-based actuation of gene expression patterning has been especially useful in two-dimensional culture or optically transparent settings. However, the inherently poor penetration of light in densely populated tissues, long exposure times needed to activate molecular switches, and corresponding challenges in patterning light delivery have limited widespread adoption of light-based patterning of gene expression in three-dimensional (3D) settings.

Heat transfer has a long industrial history, as heat is often added, removed, or moved between processes using heat exchangers, which transfer heat between fluidic networks. Recently, heat exchanger fabrication has undergone a radical shift due to developments in advanced manufacturing (e.g., 3D printing). Predating its history in industry, biological organisms have also long employed heat exchanger design principles for thermoregulation. However, existing heat exchangers are typically built from hard materials not compatible with biological systems, such as living tissues and cells.

Thus, a need exists for heat exchangers compatible with living cells and tissues that can facilitate volumetric heat patterning in artificial tissues for spatial control of biologically relevant processes, such as control of gene expression and controlled release of bioactive substances.

### BACKGROUND

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a biocompatible heat exchanger, comprising a three-dimensional thermally conductive hydrogel substrate comprising at least one fluid-perfusable channel, wherein the at least one fluid-perfusable channel comprises an inlet, an outlet, and a flow region in fluid communication with the inlet and the outlet and configured to contain a flow of a fluidic medium; and one or more heat-inducible elements configured to generate one or more biological signals when heated above or cooled below a threshold temperature.

In some embodiments, the one or more heat-inducible elements are cells genetically modified to comprise a heat inducible promoter or enhancer operatively linked to a gene of interest. In some embodiments, the one or more heat inducible promoters or enhancers is selected from the group consisting of a heat shock protein promoter, an RNA thermometer promoter, a transient receptor potential cation channel (TRPV) promoter, phage lambda pL promoter, phage lambda pR promoter, HSPB, HSP16F, HSPA1A, HSPA1B, HSPA2, and Gal80-intein.

In some embodiments, the one or more heat-inducible elements are nanoparticles comprising one or more bioactive moieties or liposomes encapsulating one or more bioactive moieties. In some embodiments, the biocompatible heat exchanger comprises a plurality of cells.

In some embodiments, the flow region is linear. In some embodiments, the flow region is non-linear. In some embodiments, the flow region comprises at least one first multifurcation downstream from the inlet, at least one first recombination upstream from outlet, and a plurality of second channels fluidly connecting the first multifurcation to the first recombination. In some embodiments, the one or more of the second channels comprises at least one second multifurcation downstream from the first multifurcation, at least one second recombination upstream from the first recombination, and a plurality of third channels fluidly connecting the second multifurcation to the second recombination.

In some embodiments, the plurality of second channels are interconnected into a grid architecture, a spherical architecture, a cubed architecture, or a rectangular cuboid architecture.

In some embodiments, the biocompatible heat exchanger comprises a plurality of fluid-perfusable channels, wherein at least two of the plurality of fluid-perfusable channels are not in fluid communication.

In some embodiments, the at least one fluid-perfusable channel comprises a valve configured to controllably regulate flow of fluid in the channel.

In some embodiments, the at least one fluid-perfusable channel has a variable diameter along its length.

In another aspect, the disclosure provides a system, comprising a heat exchanger of the disclosure and a pump configured to controllably perfuse fluidic medium into at least one inlet of the at least one fluid-perfusable channel. In some embodiments, the system comprises a plurality of the heat exchangers and one or more pumps.

In some embodiments, the system further comprises a controllable heating element configured to control the temperature of the fluidic medium. In some embodiments, the system further comprises a detector element configured to measure heat in the artificial tissue. In some embodiments, the detector element comprises an infrared camera, a thermocouple, a thermistor, a thermochromic ink, thermochromic dyes, or a combination thereof.

In another aspect, the disclosure provides an artificial tissue comprising a biocompatible heat exchanger of the disclosure.

In another aspect, the disclosure provides an artificial tissue configured for thermofluidic control of gene expression, comprising:

a biocompatible three-dimensional hydrogel substrate comprising at least one fluid-perfusable channel, wherein the at least one fluid-perfusable channel comprises an inlet port, an outlet port, and a flow region in fluid communication with the inlet and the outlet ports and configured to contain a flow of a fluidic medium; and

a plurality of cells genetically modified to comprise a heat inducible promoter or enhancer operatively linked to a gene of interest.

In another aspect, the disclosure provides a method of controlling gene expression in a three-dimensional space, comprising:

providing a plurality of genetically modified cells comprising a heat inducible promoter or enhancer operatively linked to a gene of interest in a three dimensional hydrogel substrate, wherein the three dimensional hydrogel substrate comprises at least one fluid-perfusable channel, wherein the at least one fluid-perfusable channel comprises an inlet port, an outlet port, and a flow region in fluid communication with the inlet and the outlet ports and configured to contain a flow of a fluidic medium; and

perfusing a sufficient volume of a heated fluid into the at least one fluid-perfusable channel through the at least one inlet to activate expression of the gene of interest.

In some embodiments, the genetically modified cells proximal to the fluid-perfusable channel express the gene of interest at a higher rate than genetically modified cells distal to the fluid-perfusable channel.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1G depict thermofluidic heating in exemplary 3D bioprinted hydrogels. FIG. 1A is a schematic of thermofluidic workflow. A biocompatible fluid flows around a power supplied heating element to pre-heat the fluid prior to entry in perfusable channel networks within hydrogel tissue constructs laden with heat-sensitive cells. During perfusive heating, hydrogel temperature is continuously monitored using an infrared camera. FIGS. 1B-1G depict exemplary tissues with various channel architectures. FIG. 1G shows that perfusable channel networks of varying spatial geometries can be bioprinted within biocompatible 3D hydrogels (as shown in FIGS. 1B-1F). Top panel, hydrogels channels infused with tonic water fluoresce when imaged under ultraviolet backlight. Bottom panel, Infrared thermography of heat-perfused hydrogels demonstrates that during perfusion heat traces the path of fluid flow and dissipates into the bulk hydrogel (scale bars, 5 mm).

FIGS. 2A-2F depict profile characterization in perfused hydrogels. FIG. 2A is a photograph of a single-channel bioprinted hydrogel used for initial thermal characterization; Scale bar, 5 mm). FIG. 2B shows representative infrared images from controlled perfusion of heated fluid through the channel over time (left; scale bars, 5 mm). FIG. 2C shows representative finite-element modeling images depicting steady-state predictions on the surface of perfused hydrogels at varying flow rates and constant heater power (left). Computational modeling predicts that flow rate can achieve maximal hydrogel temperatures in the mild hyperthermia temperature range (right, gray shading denotes mild hyperthermia range). FIG. 2D: hydrogels were experimentally perfused at 0.5- and 1.0-mL min⁻¹ flow rates and imaged using infrared thermography (scale bars, 5 mm). FIG. 2E: hydrogel temperature plotted orthogonal (x) to the flow direction at inlet and outlet positions show agreement between thermal gradients in computational and experimental measurements (computational, dashed lines; experimental, solid lines). FIG. 2F: hydrogel temperature plotted parallel (y) to flow direction demonstrates a larger temperature drop from inlet to outlet (y) during flow at 0.5 mL min⁻¹ (ΔT_{0.5}) compared to flow at 1.0 mL min⁻¹ (ΔT_{1.0}) in computational and experimental models (computational, dashed lines; experimental, solid lines; n=5, data are mean temperature±standard error, **p<0.01 by Student's t-test).

FIGS. 3A-3J demonstrate fluidic heating induces gene expression in 3D artificial tissues. HEK293T cells were engineered to express Firefly Luciferase under the HSPA6 promoter (FIG. 3A). FIG. 3B is a schematic of thermofluidic activation of encapsulated cells. FIG. 3C shows exemplary single-channel tissue used for 3D heat activation (left; scale bar, 3 mm). Transmittance image of cellularized hydrogel after printing (middle; scale bar, 500 μm). HEK293T cells in bioprinted tissues stained with Calcein AM (“live”,) and Ethidium Homodimer (“dead”, right, scale bar, 200 μm). FIG. 3D shows representative infrared images of thermofluidic perfusion in single-channel hydrogels. (scale bars, 2 mm) FIG. 3E demonstrates that hydrogel temperatures are tuned by changing heater power at constant flow rate (n=3, mean temperature±standard error) FIG. 3F shows representative bioluminescence images of hydrogels (top; scale bars, 2 mm) and intensity traces at three positions (A, B, C) across the width (x) of the hydrogel after 30 min of perfused heating FIG. 3G shows fold change in bioluminescence after 30 minutes of heating relative to 25° C. controls FIG. 3H shows representative bioluminescence images of hydrogels (top; scale bars, 2 mm) and intensity traces after 60 min of perfused heating (bottom; scale bars, 2 mm). FIG. 3I shows fold change in bioluminescence after 60 minutes of heating which demonstrates a temperature-dependent dosage response in gene expression. (G and I, n=3, mean fold luminescence±standard error, *p<0.05, **p<0.01 by One-Way ANOVA followed by Dunnett's multiple comparison test). FIG. 3J is a temperature-expression response curve (black) which shows mean bioluminescent radiance across temperature, shaded regions (gray) indicate±standard deviation, n=3

FIGS. 4A-4D demonstrate that exemplary heated perfusion of complex network architectures localizes gene expression in space and time. FIG. 4A shows heat exchanger inspired designs for various flow directions, fluid temperatures, and channel architectures (schematics, left and center). Representative thermal (middle) and bioluminescent (right) images demonstrate spatial tunability of thermal and gene expression patterning (scale bars, 5 mm.). FIG. 4B is a photographic image of four-armed clock-inspired hydrogel used for dynamic activation (top; channel filled with red dye). Each inlet is assigned to a local region (A-D). Schematic shows the spatial and dynamic heating pattern for the four-day study (bottom). FIG. 4C shows representative infrared (top) and bioluminescence expression (bottom) images for dynamic hydrogel activation at each day during the time course. FIG. 4D shows quantification of local bioluminescent signal from regions of interest corresponding to each day of heating. Across all four days, regions corresponding to perfused arms had higher bioluminescent signals than non-perfused arms (n=5, data are mean luminescence±standard error, *p<0.05, **p<0.01 by one-way ANOVA followed by Tukey's post-hoc test).

FIGS. 5A-5C demonstrate that HEAT gene patterning is maintained after tissue implant in vivo. FIG. 5A shows exemplary artificial tissues with embedded heat-inducible fLuc HEK293T cells that received 44° C. thermofluidic heating (Channel Heat, n=5), 44° C. global heating (Bulk Heat, n=3) or remained at 37° C. (No Heat, n=3) for 1 hour before immediate implantation into athymic mice. FIG. 5B demonstrated that bioluminescence from implanted hydrogels (dashed lines) showed regional specific signal only in channel heated hydrogels. FIG. 5C demonstrates that average line profiles (top) across the width (x) of the hydrogel for inlet, middle, and outlet positions show only channel heated gels induced a spatially coordinated response that was statistically significant (bottom) between the center (Position B) and edges of the hydrogel (Position A, C) (Channel Heat, n=5; Bulk Heat; n=3, No Heat n=3; data are mean luminescence±standard error, **p<0.01, by one-way ANOVA

FIGS. 6A-6G demonstrate thermofluidic Wnt regulation in engineered HEK293T and HepaRG cells. FIG. 6A is a schematics of lentiviral constructs (left) and thermofluidic HEK293T tissue experiments (right). FIG. 6B is a transmittance image of cellularized construct after printing (left; Zones indicated by dashed lines). Infrared image of construct during heating (right; scale bars, 1 mm). FIG. 6C shows mCherry-positive HEK293T cells in printed tissues (left; scale bars, 1 mm). Images of thermofluidically heated Wnt2 constructs after immunostaining for V5 tag (co-expressed with Wnt2; right; scale bars, 200 μm, images taken near the tissue's channel and periphery as indicated by insets). FIG. 6D shows that Wnt family genes were upregulated in Zone 3 of thermofluidically perfused gels compared to controls. (n=4, mean fold change±standard error, *p<0.05, **p<0.01 by two-way ANOVA followed by Tukey's multiple comparison test). FIG. 6E shows differentiated HepaRG cells that were engineered with a heat-inducible R-spondin 1 (RSPO1) construct (schematic, top) and printed in single-channel hydrogels (photograph, left; scale bars, 1 mm.) After heating (Infrared), HepaRGs remained viable in printed constructs (Calcein; scale bar, 200 μm). FIG. 6F shows thermofluidically heated RSPO-1 HepaRG hydrogels that were dissected into Zones 1, 2, 3 based on distance from the heat channel for RT-qPCR analysis at 1-, 24- and 48-hours post-heating. Expression fold change was normalized to no heat control samples. qPCR analysis of RSPO-1 across dissected zones (n=5-10, data are mean fold change±standard error, *p<0.05 by one-way ANOVA followed by Tukey's multiple comparison test). FIG. 6G shows RT-qPCR analysis of pooled RNA across all zones at each time point for pericentral associated genes Glutamine Synthetase, CYP1A2, CYP1A1, CYP2E1, and CYP3A4, and periportal/midzonal genes Arg1 and E-cadherin (n=15-30, data are mean fold change±standard error, **p<0.01, *p<0.05 by one-way ANOVA followed by Tukey's multiple comparison test). Photo Credit: Daniel Corbett, University of Washington

### DETAILED DESCRIPTION

The present disclosure discloses compositions, systems, and methods for a thermofluidic approach to spatially control cellular gene expression and other bioprocesses in engineered hydrogels.

In one aspect, provided herein is a biocompatible heat exchanger, comprising a three-dimensional thermally conductive hydrogel substrate. The substrate comprises at least one fluid-perfusable channel with an inlet, an outlet, and a flow region in fluid communication with the inlet and the outlet and which is configured to contain a flow of a fluidic medium. In some embodiments, the substrate also comprises one or more heat-inducible elements configured to generate one or more biological signals when heated above or cooled below a threshold temperature.

Hydrogels are three-dimensional polymeric networks filled with water that can mimic biological tissue environments. Any suitable biocompatible hydrogels can be included in the heat exchangers of the disclosure. As used herein, “biocompatible” refers to materials that are favorable to the immune system of the host in which the hydrogel is implanted. In some embodiments, the hydrogels resist protein adsorption which is believed to be one of the triggers of immune response. In some embodiments, the hydrogel is a hydrogel compatible with 3D artificial tissue printing. In some embodiments, the hydrogels have water content and elastic moduli similar to those of the human body tissues. Typically, the hydrogels can include synthetic polymers (e.g. polyethylene glycol (PEG) derivatives, polyacrylamide (PAA), polydimethylsiloxane (PDMS)), natural polymers (e.g. collagen, gelatin, alginate, hyaluronic acid (HA), and chitosan), or combinations thereof. In some embodiments, the hydrogels can comprise hyaluronic acid (HA), poly(HEMA), polymers of PEG derivatives of acrylic or methacrylic acid (e.g., PEGDA or PEGDMA), and the like. The gelation of hydrogels can be achieved by either physical or chemical crosslinking methods. In some embodiments, the hydrogels are chemically crosslinked. The hydrogels of the heat exchangers of the disclosure are thermally conductive. In some embodiments, the thermal conductivity of the hydrogels can be further increased by associating (e.g., by crosslinking) conductive materials, such as metal nanoparticles, with the polymer backbone.

The one or more heat-inducible elements include heat-inducible nanoparticles, liposomes, cells, bacteria, and the like and combinations thereof. In some embodiments, the one or more heat-inducible elements are nanoparticles that are coupled to an external field that efficiently undergo thermal induction to induce biological effects directly by hyperthermia or by secondary effects stemming from hyperthermic response (i.e. gene expression). Non-limiting examples of such nanoparticles are disclosed in Lee, J. H., Jang, J. T., Choi, J. S., Moon, S. H., Noh, S. H., Kim, J. W., & Cheon, J. (2011). Exchange-coupled magnetic nanoparticles for efficient heat induction. Nature Nanotechnology, 6(7), 418-422, the disclosure of which is incorporated herein by reference. In some embodiments, the one or more heat inducible elements are nanoparticles that are genetically encoded with a heat-sensitive receptor that can transduce a heat signal into a variety of other stimuli (e.g., mechanical, chemical, optical). Non-limiting examples of such nanoparticles are disclosed in Stanley, S. A., Sauer, J., Kane, R. S., Dordick, J. S., & Friedman, J. M. (2015). Remote regulation of glucose homeostasis in mice using genetically encoded nanoparticles. Nature medicine, 21(1), 92-98, the disclosure of which is incorporated herein by reference. In some embodiments, the one or more heat-inducible elements are temperature-sensitive liposomes that comprise and can release a biological or chemical payload upon threshold heat activation. Non-limiting examples of such nanoparticles are disclosed in Needham, D., & Dewhirst, M. W. (2001). The development and testing of a new temperature-sensitive drug delivery system for the treatment of solid tumors. Advanced drug delivery reviews, 53(3), 285-305, the disclosure of which is incorporated herein by reference.

In some embodiments, the one or more heat-inducible elements are cells genetically modified to comprise a temperature-inducible promoter or enhancer operatively linked to a gene of interest expression of which can be activated upon threshold heat activation. Any suitable heat or temperature-inducible promoters or enhancers can be used in the heat exchanger compositions disclosed herein. In some embodiments, the heat or temperature-inducible promoters or enhancers can be selected from the group consisting of a heat shock protein promoter, an RNA thermometer promoter (i.e., a promoter naturally linked to the RNA thermometer or a promoter that the RNA thermometer binds to after heat induced change in tertiary structure), a transient receptor potential cation channel (TRPV) promoter, phage lambda pL promoter, phage lambda pR promoter (e.g., a lambda promoter that require a heat labile repressor cI857), HSPB, HSP16F, HSPA1A, HSPA1B, HSPA2, Gal80-intein, or a combination thereof. In some embodiments, the genetically engineered cells comprise a temperature-sensitive intein, such as one of the inteins disclosed in Zeidler, M., Tan, C., Bellaiche, Y. et al. Temperature-sensitive control of protein activity by conditionally splicing inteins. Nat Biotechnol 22, 871-876 (2004), the disclosure of which is incorporated herein by reference.

In some embodiments, the heat-inducible elements are configured to generate one or more biological signals. Biological signals include expression of a protein of interest, release of a biologically relevant molecule from a nanoparticle or a liposome encapsulating it, and the like. Biologically relevant molecules include proteins, peptides, lipids, carbohydrates, nucleic acids (e.g., RNA, DNA), and small molecules (e.g., pharmaceutical agents).

In some embodiments, in addition to one or more heat-inducible elements, the biocompatible heat exchanger comprises a plurality of cells that have not been genetically engineered to comprise a heat inducible promoter or enhancer. In some embodiments, the biocompatible heat exchanger comprises two or more types of heat-inducible elements, e.g., a liposome and a cell genetically modified to comprise a temperature-inducible promoter or enhancer operatively linked to a gene of interest. In some embodiments, the biocompatible heat exchanger is inside an artificial tissue.

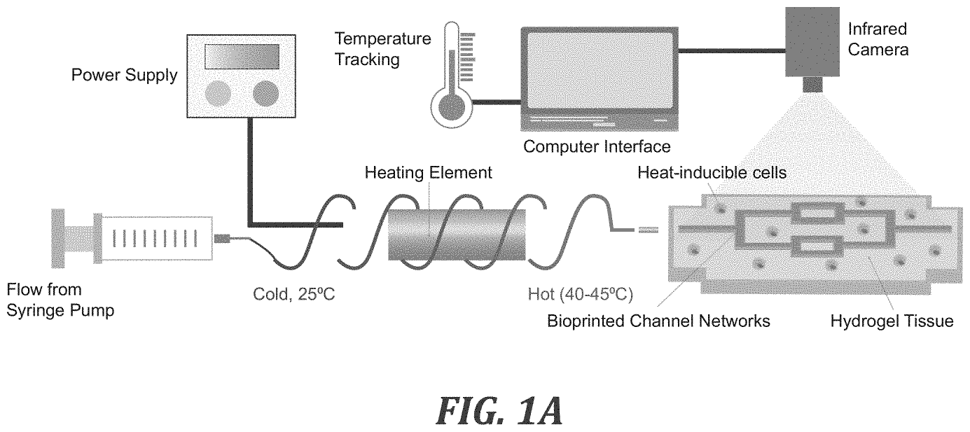

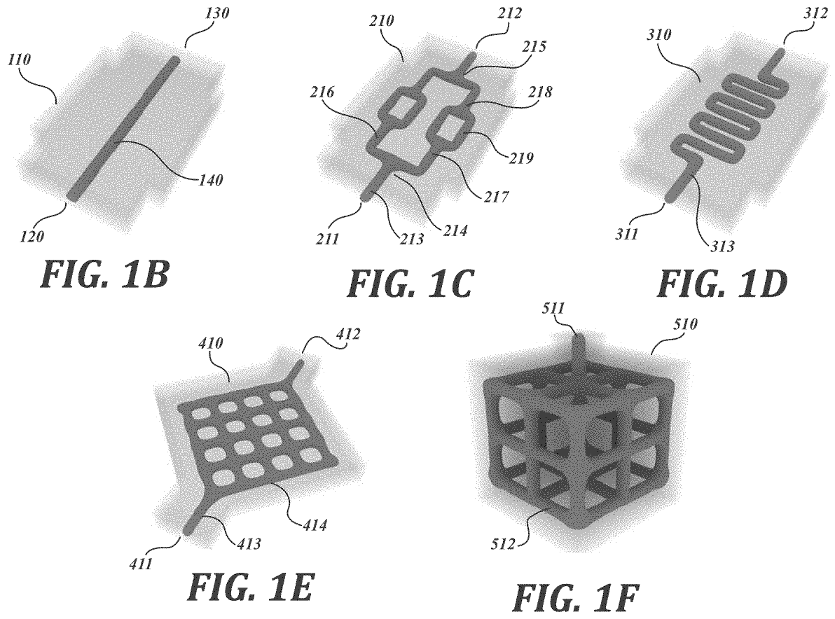

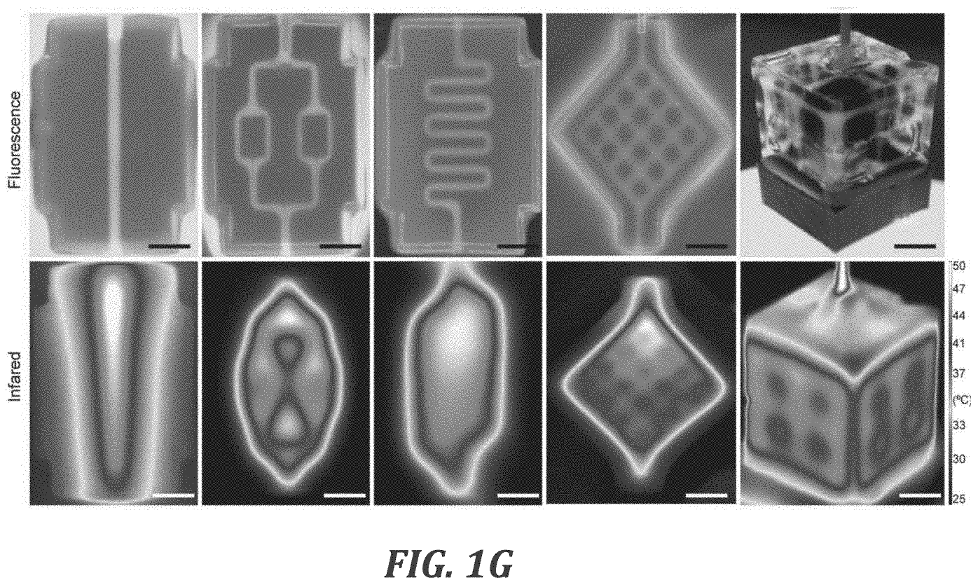

The one or more channels of the biocompatible heat exchanger can have any suitable geometry that allows perfusion of a biologically compatible perfusive fluid through the hydrogel. In some embodiments, the flow region can have axial or linear geometry. One such exemplary channel is shown in FIG. 1B, depicting a hydrogel substrate (110) in which a channel has a linear flow region (140) connecting the inlet (120) and the outlet (130). In some embodiments, the flow region is non-linear, for example, it can have a serpentine architecture. One such exemplary channel is shown in FIG. 1D, depicting a hydrogel substrate (310) in which a channel has a non-linear (serpentine) flow region (313) connecting the inlet (311) and the outlet (313).

In some embodiments, the flow region can have a branched architecture. In some embodiments, the flow region comprises at least one first multifurcation downstream from the inlet, at least one first recombination upstream from outlet, and a plurality of second channels fluidly connecting the first multifurcation to the first recombination, i.e., the flow region can have a branched architecture. In some embodiments, each second channel can comprise additional branching, i.e., each channel can have a second multifurcation and be split into multiple third channels that recombine into the second channel, and so on. In some embodiments, the one or more of the second channels comprises at least one second multifurcation downstream from the first multifurcation, at least one second recombination upstream from the first recombination, and a plurality of third channels fluidly connecting the second multifurcation to the second recombination. An exemplary channel that has two branchings is shown in FIG. 1C, which depicts a hydrogel substrate (210) with a channel in which a flow region (213) connecting the inlet (211) and the outlet (213) comprises a first bifurcation (214) and a first recombination (215) splitting the channel into two second channels (216), wherein each second channel comprises a second bifurcation (217) splitting each second channel into two third channels (219) that recombine at the second recombination (218). In some embodiments, the flow region can comprise multiple branchings.

In some embodiments, the plurality of second channels can be interconnected into a grid architecture, a spherical architecture, a cuboid architecture, or a rectangular cuboid architecture. Any configuration that allows a flow of perfusion fluid through the heat exchange is possible and can be configured to the particular purpose. An exemplary channel with a grid architecture of the flow region is shown in FIG. 1D, which depicts a hydrogel substrate (410) with a channel (413) having an inlet (411) and an outlet (412) and a flow region having a grid architecture (414). Another exemplary channel, a channel with a cuboid flow region architecture is shown in FIG. 1E, which depicts a hydrogel substrate (510) with a channel having a channel (511) with a flow region having a cuboid architecture (512).

In some embodiments, the flow regions can comprise a combination of the flow region architectures disclosed above, for example, a cuboid region can be followed by a non-linear region. In some embodiments, the biocompatible heat exchanger comprises a plurality of fluid-perfusable channels, wherein at least two of the plurality of fluid-perfusable channels are not in fluid communication. In some embodiments, the biocompatible heat exchanger comprises a plurality of fluid-perfusable channels, wherein at least two of the fluid-perfusable channels have a different architecture of the flow region. In some embodiments, the biocompatible heat exchanger comprises a plurality of fluid-perfusable channels wherein the at least one fluid-perfusable channel has a variable diameter along its length. In some embodiments, all fluid-perfusable channels have a variable diameter along their length. In some embodiments, all fluid-perfusable channels have a fixed diameter along their length.

In some embodiments, at least one fluid-perfusable channel comprises a valve configured to controllably regulate flow of fluid in the channel. In some embodiments, all fluid-perfusable channels comprises a valve configured to controllably regulate flow of fluid in the channel.

Typically, the fluidic medium that can be used with the heat exchangers of the disclosure, is a biocompatible fluid. Non-limiting examples of such fluids include water, saline, and biological fluids such as blood, plasma, or serum. Synthetic biocompatible fluids, such as synthetic blood substitutes, can be also used.

A used herein, the “threshold temperature” means the temperature below or above which the one or more of the heat-inducible elements configured to generate one or more biological signals will begin to generate the corresponding signal. For example, in some instances, when the heat exchanger comprises a plurality of cells with a heat-activated promoter operably linked to a gene of interest, at least a portion of the cells will begin to express the protein or peptide encoded by the gene of interest when heated above the threshold temperature. In some embodiments, the threshold temperature is a temperature sufficient to activate expression of the gene of interest. In some embodiments, the threshold temperature is a human body temperature (e.g., temperature of about 36° C. to about 43° C.). In some embodiments, the temperature is a temperature above a human body temperature, e.g. a temperature above about 43° C. In some embodiments, the threshold temperature is a temperature useful for performing a PCR amplification of a nucleic acid using a thermostable polymerase (e.g., a Taq polymerase), such as a temperature of about 60° C. to about 95° C.; in such instances, the heat exchangers can be used as an in situ nucleic acid delivery device. In some embodiments, the threshold temperature is a temperature that results in changes of one or more material property of the hydrogel, for example, when the hydrogel is a temperature-responsive hydrogel such as those disclosed in Liang, R., Yu, H., Wang, L., Lin, L., Wang, N., & Naveed, K. U. R. (2019). Highly Tough Hydrogels with the Body Temperature-Responsive Shape Memory Effect. ACS Applied Materials & Interfaces, 11(46), 43563-43572, the disclosure of which is incorporated herein by reference.

In some embodiments, the heat-inducible elements, e.g., genetically modified cells, can be evenly interspersed throughout the hydrogel. In some embodiments, the heat-inducible elements can populate areas immediately adjacent or proximal to the one or more fluid-perfusable channels. In some embodiments, the heat-inducible elements can be attached to the inner walls of the one or more fluid-perfusable channels. In some embodiments, the genetically modified cells proximal to the fluid-perfusable channel express the gene of interest at a higher rate than genetically modified cells distal to the fluid-perfusable channel.

In another aspect, the disclosure provides a system comprising one or more biocompatible heat exchangers disclosed herein and a pump configured to controllably perfuse fluidic medium into at least one inlet of the at least one fluid-perfusible channel. The pump can be connected to the one or more heat exchanges in any suitable manner. In some embodiments, the pump can be connected directly to the heat exchanger, and in some embodiments, the system can comprise a pipe or tubing connecting the pipe and the one or more heat exchangers.

The systems of the disclosure can further comprise other elements. In some embodiments, the system comprises a controllable heating element configured to control the temperature of the fluidic medium. Examples of suitable heating elements include voltage-controlled resistance heater, Peltier element, etc.

In some embodiments, the system further comprises one or more detector elements configured to measure heat in the artificial tissue, e.g., an infrared camera, a thermocouple, a thermistor, a thermochromic ink, thermochromic dyes, or a combination thereof.

In some embodiments, other elements of the systems can include one or more computer elements configured for processing, storing, and/or displaying heat parameters in the tissue. In some embodiments, the system can include one or more controller elements to regulate the heating and/or perfusion rates, potentially based on feedback from the detector element.

In another aspect, the disclosure provides an artificial tissue comprising one or more of the biocompatible heat exchangers described above. In some embodiments, the artificial tissue comprises one or more types of cells.

In some embodiments, the artificial tissue is configured for thermofluidic control of gene expression and comprises a biocompatible three-dimensional hydrogel substrate comprising at least one fluid-perfusable channel, wherein the at least one fluid-perfusable channel comprises an inlet port, an outlet port, and a flow region in fluid communication with the inlet and the outlet ports and configured to contain a flow of a fluidic medium; and a plurality of cells genetically modified to comprise a heat inducible promoter or enhancer operatively linked to a gene of interest.

In another aspect, the disclosure provides a method of controlling gene expression in a three-dimensional space, comprising:

providing a plurality of heat-inducible elements in a three dimensional hydrogel substrate, wherein the three dimensional hydrogel substrate comprises at least one fluid-perfusable channel, wherein the at least one fluid-perfusable channel comprises an inlet port, an outlet port, and a flow region in fluid communication with the inlet and the outlet ports and configured to contain a flow of a fluidic medium; and

perfusing as sufficient volume of a heated fluid into the at least one fluid-perfusable channel through the at least one inlet.

In some embodiments, the heat-inducible elements are genetically modified cells comprising a heat inducible promoter or enhancer operatively linked to a gene of interest. In some embodiments, the genetically modified cells proximal to the fluid-perfusable channel express the gene of interest at a higher rate than genetically modified cells distal to the fluid-perfusable channel.

Unless specifically defined herein, all terms used herein have the same meaning as they would to one skilled in the art of the present invention. The use of the term “or” in the claims is used to mean “and/or” unless explicitly indicated to refer to alternatives only or the alternatives are mutually exclusive, although the disclosure supports a definition that refers to only alternatives and “and/or.” The words “a” and “an,” when used in conjunction with the word “comprising” in the claims or specification, denote one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element. Additionally, the words “herein,” “above,” and “below,” and words of similar import, when used in this application, shall refer to this application as a whole and not to any particular portions of the application. As used herein, the term “about” includes ±5% of the stated value.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these components etc. may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

All publications cited herein and the subject matter for which they are cited are hereby specifically incorporated by reference in their entireties.

The following examples are provided to illustrate certain particular features and/or embodiments of the disclosure. The examples should not be construed to limit the disclosure to the particular features or embodiments described.

### Examples

The following examples describe exemplary biocompatible heat exchangers, artificial tissues, systems comprising the same, and thermofluidic methods for mesoscale spatiotemporal control of gene expression. The methods exploits volumetric fluid-based heat transfer, which is referred to herein as “Heat Exchangers for Actuation of Transcription” (HEAT), (FIG. 1A). HEAT leverages the use of projection stereolithography bioprinting technology to fabricate topologically complex fluidic channels of user-defined geometries in hydrogels (FIG. 1B, top and middle). 3D printed hydrogels are laden with genetically engineered heat-inducible cells during the printing process (FIG. 1A). Encased channel networks are perfused with precisely heated fluid from a power-supplied heating element. During perfusion, tissue temperature is monitored in real-time using an infrared camera (FIG. 1A). The thermofluidic perfusion facilitates heat transfer from the channels into the bulk hydrogel and enables architectural heat patterning in hydrogels (FIG. 1B, bottom).

Materials and Methods

Materials and Photopolymer Synthesis.

Poly(ethylene glycol) diacrylate (PEGDA; 6000 Da) and Lithium phenyl-2,4,6-trimethylbenzoylphosphinate (LAP) were prepared as previously described. Gelatin methacrylate (GelMA) was synthesized as previously described, with slight modifications. Methacrylic anhydride was added dropwise to gelatin dissolved in carbonate-bicarbonate buffer at 50° C. for 3 hours, followed by precipitation in ethanol. The precipitate was allowed to dry, dissolved in PBS, frozen at −80° C., then lyophilized for up to 1 week. GelMA was stored at −20° C. until use. Tartrazine (Sigma T0388, St. Louis, Mo., USA) was added to prepolymer solutions as a photoabsorber to increase print resolution. Prepolymer mixtures for all cellular studies contained 7.5 wt % 6K PEGDA, 7.5 wt % GelMa with 17 mM LAP and 1.591 mM tartrazine. For characterization of heat transfer with respect to gel density, the overall polymer weight % was varied while holding the ratio of 6K PEGDA to GelMA constant at 50:50 (ex: 20 weight %=10 wt % 6K PEGA+10 wt % GelMa).

Model Design.

Hydrogels with perfusable channel networks were designed in an open-source 3D computer graphics software Blender 2.7 (Blender Foundation, Amsterdam, Netherlands) or in Solidworks (Dassault Systemes SolidWorks Corp., Waltham, Mass.).

3D Printing.

A stereolithography apparatus for tissue engineering (SLATE) bioprinting system was used in this study. Briefly, the system contains 3 major components: 1) a Z-axis with stepper motor linear drive; 2) an open-source RepRap Arduino Mega Board (RAMBo; Ultimachine, South Pittsburgh, Tenn.) microcontroller for Z-axis control of the build platform; and 3) a projection system consisting of a DLP4500 Optical Engine with a 405 nm LED output (Wintech, Carlsbad, Calif.) connected to a laptop for photomask projection and motor control. The projector was placed in front of the Z-axis and a mirror is positioned at 45° to the projection light path to reflect projected images onto the build platform. A sequence of photomasks based on a 3D model was prepared using Creation Workshop software (http://www.envisionlabs.net/) which also controls the Z-axis movement of the build platform. Printing was achieved by curing sequential model layers of the photosensitive prepolymer. All printing was conducted in a sterile tissue culture hood. For visualization of channel networks, open channels were perfused with UV fluorescent tonic water or India ink dyes (Dr. Ph. Martin's, Oceanside, Calif.).

In-Line Fluid Heating System.

To control temperature distribution in perfused hydrogels, an in-line fluid heater was developed to pre-warm perfusate solutions prior to infusion in hydrogel channel networks. The fluid heater consists of four components: 1) an adjustable DC Power Supply (Yescom USA, Inc., City of Industry, Calif.); 2) a cylindrical cartridge heater (Uxcell, Hong Kong); 3) perfusate tubing (Peroxide-Cured Silicone Tubing, Cole Parmer, Vernon Hills, Ill.); and 4) a syringe pump (Harvard Apparatus, Holliston, Mass.). To construct the in-line fluid heater, perfusate tubing was connected to the syringe pump for flow rate control, while the cartridge heater was connected to the power supply for heating control. Perfusate tubing was then wound around the cylindrical cartridge heater, allowing for heat transfer from the heater into the flowing perfusate. The temperature of the fluid was then controlled by changing the flow rate or heater power. In all studies we used phosphate buffered saline (Fisher, Hampton, N.H.) for the perfusate solution.

Hydrogel Fluidic Connections.

To establish a fluidic connection between the heating system and hydrogel channel networks, custom-designed 3D printed perfusion chips were printed on a Makergear M2 3D printer (Makergear, Beachwood, Ohio) in consumer grade poly(lactic acid) (PLA) plastic filament. Perfusion chips were fabricated with 1) an open cavity to insert 3D bioprinted hydrogels and 2) attachment ports for fluid-dispensing nozzles. The outflow of the fluid heater was fitted with a male luer hose barb (Cole Parmer) connected to a flexible tip, polypropylene nozzle (Nordson EFD, East Providence, R.I.) and inserted into 3D printed attachment ports. Hydrogels were then inserted to perfusion chips and proper fluidic connections were ensured before beginning perfusion.

Infrared Thermography.

Fluid temperature and heat distribution were measured in perfused hydrogels by infrared thermography. Images were acquired by an uncooled microbolometer type infrared camera (FLIR A655sc, Wilsonville, Oreg.) which detects a 7.5-14.0 μm spectral response with a thermal sensitivity of <0.05° C. and analyzed for temperature values using the FLIR ResearchIR software (Wilsonville, Oreg.).

Computational Models.

Ffinite element models of perfused hydrogels in COMSOL 4.4 software (Comsol AB, Burlington Mass.) were built. Simulations were run under transient conditions using the “Conjugate heat transfer” module, and 3D printed hydrogel and housing geometries to predict the temperature distribution. The model was based on (1) forced convective heat transfer from the perfusion channel to the hydrogel volume and (2) conductive heat transfer within the hydrogel volume.

Equation for (1). Heat Transfer in a Fluid:

$$ρ {C}_{ρ} \frac{\partial T}{\partial t} + ρ {C}_{ρ} u \cdot ∇ T = {α}_{p} T ( \frac{\partial {p}_{A}}{\partial t} + u \cdot ∇ {p}_{A} ) + τ : S + ∇ \cdot ( k ∇ T ) +$$

Where ρ is the fluid density, T is the temperature, C_{ρ} is the heat capacity at constant pressure, u is the velocity field, α is the thermal expansion coefficient, p_{A} is the absolute pressure, τ is the viscous stress tensor, S is the strain rate tensor, k is the fluid thermal conductivity and Q is the heat content.

Equation for (2):

$$ρ {C}_{p} \frac{\partial T}{\partial t} = ∇ \cdot ( k ∇ T ) + Q$$

Where ρ is the hydrogel density, T is the temperature, k is the hydrogel thermal conductivity and Q is the heat content.

Material properties of both the hydrogel and perfusate were modeled as water. Heat flux boundary conditions were included to model heat loss to the ambient environment, heat transfer coefficients of 5 and 30 W/(m*K) were applied to the sides and upper boundaries of the hydrogel respectively, with an infinite temperature condition of 22.0° C. applied for all boundaries. Boundary temperature and fluid inflow conditions at the channel inlet were used to simulate the effect of changing perfusate temperature and flow rate, respectively. Model geometry was manipulated for studies on channel length and channel branching. Prescribed external temperature was varied for ambient temperature studies.

Cell Culture.

HEK293T cells were maintained in Dulbecco's modified Eagle's medium (DMEM) (Corning, N.Y., USA) supplemented with 10% (v/v) fetal bovine serum (FBS) (GIBCO), 1% (v/v) penicillin-streptomycin (GE Healthcare Life Sciences, WA, USA). Differentiated HepaRG cells (Fisher) were maintained at confluence in 6 well plates at a density of 2×10⁶ cells/well in Williams E media (Lonza, Md., USA) supplemented with 5× HepaRG™ Thaw, Plate & General Purpose Medium Supplement (Fisher), 1% (v/v) Glutamax (Fisher).

Construction of Heat-Sensitive Reporter Gene Cells.

A vector containing a 476 bp version of the human heat shock protein 6a (HSPA6) promoter driving expression of firefly luciferase (fLuc) reporter gene (Gift of Dr. Ruth Schez Shouval from the Weizmann Institute of Science) was packaged into lentivirus using helper plasmids pMDLg/pRRE (Addgene 12251), pMD2.G (Addgene 12259), and pRSV-Rev (Addgene 12253) by co-transduction into HEK293T cells. Crude viral particles were harvested after 48 hours of transduction. For viral transduction, crude lentivirus was diluted 1:20 in DMEM containing polybrene (6 μg/mL; Invitrogen), added to competent HEK293T cells in six-well tissue culture plates and incubated overnight (Corning). The next day virus-containing media was removed and replaced with fresh DMEM containing 10% FBS. After transduction cells were heat activated (see below) and flow sorted for positive GFP expression to obtain a pure cell population.

Heat Treatment.

To activate transgene expression under the HSPA6 promoter, engineered HEK293T cells were exposed to varying levels of hyperthermia in 2D and 3D. For 2D heat treatment studies, cells were seeded at 8×10⁴ cells/cm² in tissue culture plates one day prior to heat treatment. The next day tissue culture plates were exposed to indicated heat treatments in thermostatically controlled cell culture incubators. Temperature was verified with a secondary method by a thermocouple placed inside the incubator. Upon completion of heat treatment, cells were returned to a 37° C. environment and sorted or analyzed at later time points. For the luminescent transient studies, cells were lysed in TE buffer (100 mM Tris, 4 mM EDTA, pH=7.5) and stored at 4° C. until imaging. For the pulsed activation studies in FIG. S4C, cells received two heat shocks as described previously at days 0 and 3. Luminescence was quantified across days 1-4 and normalized to cell counts from tissue culture plates that were processed in parallel according to each experimental temperature. For 3D heat shock studies, cells were encapsulated and printed in 3D perfusable hydrogels (see below) one day prior to heating. 3D hydrogels were then heat perfused in a room temperature environment. Hydrogel temperature was monitored continuously with the infrared camera and small adjustments to heater power were made as necessary to maintain a stable temperature profile. During perfused heating, outlet medium was continuously discarded. Upon completion of perfused heating, hydrogels were dismounted from the perfusion chips and returned to a cell culture incubator.

Cell Encapsulation and Printing of Cell-Laden Hydrogels.

Cultured HEK293T cells were detached from tissue culture plates with 0.25% trypsin solution (Corning), counted, centrifuged at 1,000 rpm for 5 min and resuspended in liquid prepolymer (7.5 wt % 6k PEGDA, 7.5 wt % GelMA, 17 mM LAP, 1.591 mM tartrazine). For characterization of heat transfer with respect to cell density, cells were encapsulated in prepolymer mixtures at final densities from 0-24×10⁶ cells mL⁻¹ prior to printing. For HEK293T expression studies, cells were encapsulated at a final density of 6×10⁶ cells mL⁻¹. For HepaRG studies, cells were encapsulated at a final density of 2.5×10⁶ cells mL⁻¹. Printing was performed as previously described under DLP light intensities ranging from 17-24.5 mW cm⁻², with bottom layer exposure times from 30-35 s and remaining layer exposure times from 12-17.5 s. Upon print completion, fabricated hydrogels were removed from the platform with a sterile razor blade and allowed to swell in cell culture media. Hydrogels were changed to fresh media 15 min after swelling and allowed to incubate overnight. Media was replaced the following morning. We tested the viability of both HEK293T and HepaRG cells following 3D printing by incubating cell-laden hydrogels with Live/Dead viability/cytotoxicity kit reagents (Life Technologies, Carlsbad, Calif.) according to manufacturer's instructions. Fluorescence imaging was performed on a Nikon Eclipse Ti inverted epifluorescent microscope and images were quantified using ImageJ's built in particle analyzer tool (National Institutes of Health (NIH), Bethesda, Md.).

Bioluminescent Imaging.

To visualize the magnitude and spatial localization of heat-induced luciferase expression, bioluminescence imaging was performed on heated cells and hydrogels using the IVIS Spectrum imaging system (PerkinElmer, Waltham, Mass.). Immediately prior to bioluminescence imaging, cell culture media was changed to media containing 0.15 mg/mL D-Luciferin (PerkinElmer) and images were taken every 2 min until a bioluminescent maximum was reached. Images were analyzed using Living Image software (PerkinElmer). Luminescent imaging was performed from a ‘top-down’ view (perspective orthogonal to hydrogel channel axis) for most studies. For cross-sectional images in FIG. S8, hydrogels were manually sliced, incubated in luciferin containing media and imaged under cross section view (perspective parallel to hydrogel channel axis).

Pixel-to-Pixel Temperature-to-Expression Correlation.

Data for the expression vs. temperature plot was obtained by aligning thermal and bioluminescent images using MATLAB. To align the images, four reference points corresponding to the corners of the hydrogel were manually selected on both thermal and bioluminescence images. Then, an orthogonal transformation was performed on each image to align the corners of the hydrogel, after which the areas outside the selection were cropped. Pixel values from each image were then plotted against each other to produce the expression vs. temperature plot.

In Vivo Implantation of HEAT-Modulated Artificial Tissues.

Heat-inducible cells were generated as previously described and embedded into 3D-printed artificial tissues with single channels before being placed at 37° C. overnight. The next day, artificial tissues received either thermofluidic heat stimulation via flow of 44° C. biocompatible fluid at 1.0 mL min⁻¹ for 60 minutes (n=5), global heat stimulus by being placed in a 44° C. tissue culture incubator for 60 minutes (n=3) or were maintained in a 37° C. tissue culture incubator (n=3). The artificial tissues were then immediately implanted subcutaneously on the ventral side of female NCr nude mice aged 8-12 weeks old (Taconic). 24 hours after implantation, mice were anesthetized and injected with luciferin (15 mg/mL, Perkin Elmer, Waltham, Mass.). Bioluminescence was then recorded via the IVIS Spectrum imaging system (PerkinElmer). For 3D images, a custom 3D imaging unit developed by Alexander D. Klose and Neal Paragas (InVivo Analytics, New York, N.Y.) was used. Briefly, anesthetized mice were placed into body-fitting animal shuttles and secured into the custom 3D imaging unit that utilizes a mirror gantry for multiview bioluminescent imaging. Collected images were then compiled and overlaid onto a standard mouse skeleton for perspective.

Spatial Analysis of In Vivo IVIS Images.

Line profiles in the x-direction across the inlet, middle and outlet of 2D IVIS projection images from artificial gels were generated using Living Systems Software (PerkinElmer, Waltham, Mass.). The three line profiles (inlet, middle and outlet) from each artificial tissue were then averaged together with the average line profiles from the other artificial gels within each respective group (experimental group, n=5; positive control group, n=3; negative control group, n=3). The average line profile of each group was then plotted, and average radiance values from positions 0.75 cm from the center of the channel (denoted positions A and C) were then statistically compared to the average radiance value at the center of the channel (Position B) within each group by one-way ANOVA.

Generation of Wnt Constructs and Cells.

Lentiviral constructs in which the HSPA6 promoter drives a Wnt family gene were subcloned using Gibson assembly by the UW BioFab facility. Human beta-catenin pcDNA3 was a gift from Eric Fearon (Addgene plasmid #16828; http://n2t.net/addgene:16828; RRID:Addgene 16828; A. D. Klose, N. Paragas, Automated quantification of bioluminescence images. Nat. Commun. 9, 4262 (2018), the disclosure of which is incorporated herein by reference). Active Wnt2-V5 was a gift from Xi He (Addgene plasmid #43809; http://n2t.net/addgene:43809; RRID:Addgene_43809; F. T. Kolligs, G. Hu, C. V. Dang, E. R. Fearon, Neoplastic Transformation of RK3E by Mutant β-Catenin Requires Deregulation of Tcf/Lef Transcription but Not Activation of c-myc Expression. Mol. Cell. Biol. 19, 5696-5706 (1999), the disclosure of which is incorporated herein by reference). RSPO1 was subcloned using a cDNA clone plasmid. (Sino Biological, Beijing, China). All plasmids contained a downstream cassette in which a constitutive promoter (spleen focus-forming virus, SFFV) drives the reporter gene mCherry (gift from Dr. Gabriel A. Kwong, Georgia Institute of Technology). Lentivirus was generated by co-transfection of HEK293 Ts or HepaRGs with HSPA6→Wnt transfer plasmids with 3^{rd} generation packaging plasmids (pMDLg/pRRE, pMD2.G, pRSV-REV) in DMEM supplemented with 0.3% Xtreme Gene Mix (Sigma). Crude virus was harvested starting the day after initial transfection for four consecutive days. For viral transduction, HEK293 Ts at 70% confluency and HepaRGs at 100% confluency were treated with crude virus containing polybrene (8 μg/mL, Sigma) for 24 hours. Five days following viral transduction, mCherry positive HEK293 Ts sorted from the bulk population by flow cytometry at the UW Flow analysis facility. HepaRGs were not sorted by flow cytometry. mCherry expression in positive HEK293T cell populations was performed using RT-qPCR.

Wnt Upregulation in HEAT-Induced Constructs.

To quantify Wnt regulator levels in HEAT treated gels, HEK293 Ts and HepaRGs for a given construct were encapsulated and heated in 3D hydrogels as previously described. ‘No heat control’ samples remained at 37° C. in tissue culture incubators until RNA isolation. 1-48 hours following heat treatment, hydrogels were manually sliced into corresponding zones (1, 2, 3) and RNA was isolated using phenol-chloroform extraction. cDNA was synthesized using the Superscript III First strand synthesis kit (ThermoFisher) and qPCR performed using iTaq Universal SYBR Green Supermix (Biorad, Hercules, Calif. on a 7900HT Real Time PCR system (Applied Biosystems, Waltham, Mass.). Primers for Wnt and housekeeping genes were designed and synthesized by Integrated DNA Technologies (Coraville, Iowa). Relative gene expression was normalized against the housekeeping gene 18s RNA calculated using the ΔΔCt method. Data are presented as the mean relative expression ±s.e.m. Data for HEK293T studies was normalized to relative expression of the Wnt target in 2D culture at 37° C. Data for HEK293T mCherry expression was normalized to 18s RNA and compared to GAPDH (also normalized to 18s RNA) expression levels. Data for HepaRG studies was normalized by relative expression of the Wnt target or pericentral/periportal gene marker to ‘No heat control’ samples.

V5 Staining/Clearing in Wnt2 HEAT Gel.

HSPA6→Wnt2N5 gels were fixed in 4% paraformaldehyde 24 hours post-heating. For staining, samples are blocked overnight at room temperature in 1% BSA+1% normal donkey serum+0.1M tris+0.3% Triton X-100 with agitation. After blocking, samples are incubated in Anti-V5 tag antibody (Abcam, ab27671) diluted 1:100 in fresh blocking buffer +5% dimethyl sulfoxide for 24 hours at 37° C. and agitation. Samples are washed, then incubated in secondary antibody diluted 1:500 in fresh blocking buffer+5% dimethyl sulfoxide overnight at 37° C. and agitation. After incubation samples are washed in PBS+0.2% Triton X-100+0.5% 1-thioglycerol three times at room temperature and agitation, changing fresh buffer every 2 hours. To begin clearing, samples are incubated in Clearing Enhanced 3D (Ce3D) (L. S. Toni et al., Optimization of phenol-chloroform RNA extraction. MethodsX 5, 599-608 (2018), the disclosure of which is incorporated herein by reference) solution at room temperature overnight with agitation protected from light. DAPI is diluted 1:500 in the Ce3D solution in order to counter stain for nuclei. To 3D image the cleared samples, the gels are placed on glass-bottom dishes and imaged overnight on an SP8 Resonant Scanning Confocal Microscope.

Statistics.

Data in graphs are expressed as the mean±standard error or mean±standard deviation, as denoted in figure legends. Statistical significance was determined using two tailed Student's t-test for two-way comparisons or one-way ANOVA or two-way ANOVA followed by Dunnett's, Sidak's or Tukey's multiple comparison test.

Thermal Characterization in Single Channel Hydrogels.

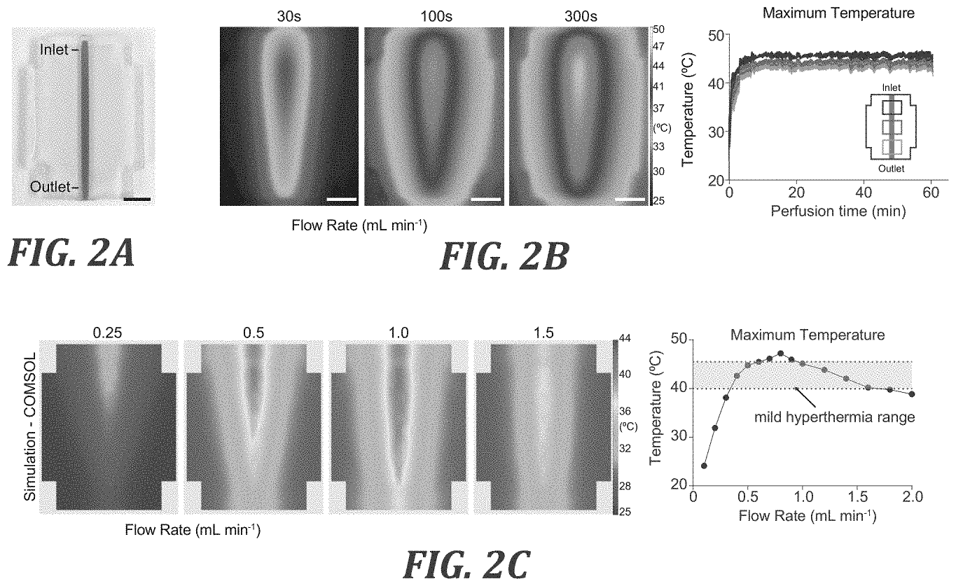

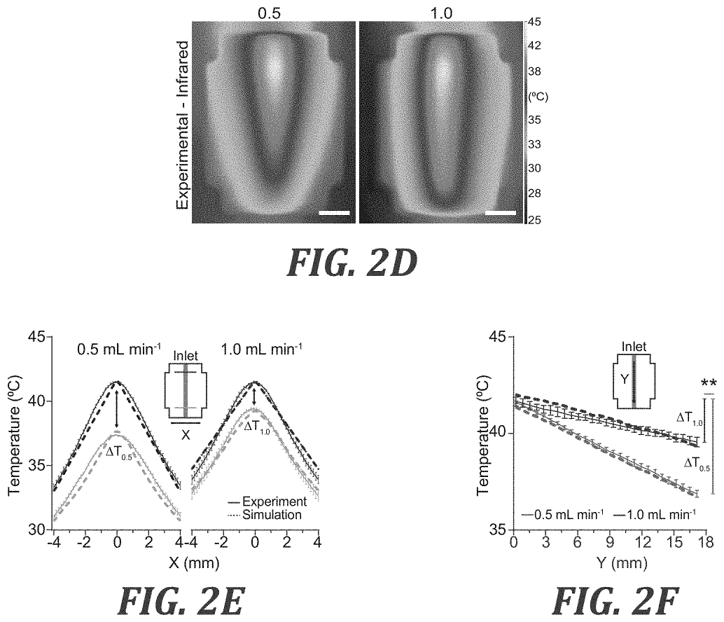

Most mammalian thermally-inducible gene switches require exposure to mild hyperthermia (39° C.-45° C.) for prolonged periods of ˜15-60 minutes to activate transcription. The inventors tested whether this approach could precisely regulate tissue temperature over prolonged periods of time by maintaining steady-state thermal profiles in perfused hydrogels. To do this, hydrogels that contained a single channel were first printed (FIG. 2A). Precisely heated fluid was then perfused through this channel while tracking hydrogel temperature in real-time using infrared thermography (FIG. 2B). Upon initiating perfusion, it was observed that hydrogel temperature underwent an initial ramp-up phase (˜5 minutes) followed by a steady-state plateau in which temperature deviated by <±0.4° C./min at three separate regions measured across the hydrogel (FIG. 2B, right).

During perfusion, heat is transferred from fluidic channels to the bulk through convection and conduction, resulting in thermal gradients throughout the bulk volume. The perfusate input temperature is known to govern the rate and magnitude of heat transfer, while fluid flow rate influences the thermal profile. To determine the relative effects of perfusate temperature and flow rate on hydrogel heating at biologically relevant temperatures, the inventors sought to develop a finite element model of heated hydrogel perfusion for mild hyperthermia that incorporated thermal and flow parameters from our heating system. To derive these parameters, flow rate was first incrementally increased over a range of heating element powers and fluid temperature was measured at the point of heater outflow (i.e., hydrogel inlet). The inventors then implemented perfusate temperature values observed from each flow rate at 13.5 W heater power into a computational model of single-channel hydrogel heating (FIG. 2C). Computational simulations predicted that hydrogel temperatures in the range for mild hyperthermia were achievable using flow rates from 0.4-1.6 mL min⁻¹, but not for slower or faster flow rates (FIG. 2C). Within this window, it was observed that flow rates 0.5- and 1.0-mL min⁻¹ produced subtle differences in the shape of thermal profiles, despite roughly equivalent input temperatures (FIG. 2C). Thus, these flow rates provided a set of conditions to further examine the effects of flow rate on heat transfer.

The inventors therefore performed experimental validation studies of perfused single channel hydrogels at 0.5- or 1.0-mL min⁻¹ and analyzed the steady-state thermal profiles from infrared images (FIG. 2D). Experimental temperature measurements (solid lines) and computational simulation predictions (dashed lines) showed agreement when measured both orthogonal (FIG. 2E), and parallel (FIG. 2F) to channel flow. Both physical measurements and simulations demonstrated thermal gradients in the hydrogel. Temperature along the channel was better maintained under flow at 1.0 mL min⁻¹ compared to flow at 0.5 mL min⁻¹ (FIGS. 2E and 2F, **p<0.01), and flow at 0.5 mL min⁻¹ promoted more heat transfer at the channel inlet. Importantly, addition of cells to single-channel hydrogels did not affect temperature profile after thermofluidic perfusion, nor did differences in hydrogel weight percent in ranges commonly used for 3D printing of cellularized hydrogels (i.e., 10-20 wt %; B. Grigoryan et al., Multivascular networks and functional intravascular topologies within biocompatible hydrogels. Science. 364, 458-464 (2019), the disclosure of which is incorporated hereinby reference). Stiffer hydrogel formulations (i.e., 25 wt %) did exhibit different temperatures at the hydrogel edge, though such formulations are less commonly used for bioprinting due to their limited support of cell viability.

These findings led to further computational exploration of the potential spatial design space for a single-channel system. To do this, the inventors assessed how varying channel length and ambient temperature affect the thermal profile in the model described herein. Predictions showed that single channels up to 30 mm long achieved hyperthermic temperatures (40-45° C.) along their entire length, with outlet temperatures falling out of the hyperthermic range at greater lengths. Spatial heat distribution was not significantly affected within the ambient temperature range used in our studies here (20-22° C.), but more substantive increases in ambient temperature (e.g., to 30-37° C.) produced wider spatial gradients in hyperthermic range. Taken together, these studies showed that the rules of heat transfer could be leveraged to predict thermal spatial profiles in perfused hydrogels and that these profiles could be finely tuned by varying parameters such as flow rate, channel length, and input and ambient temperature.

Generation and Characterization of Heat-Inducible Cells

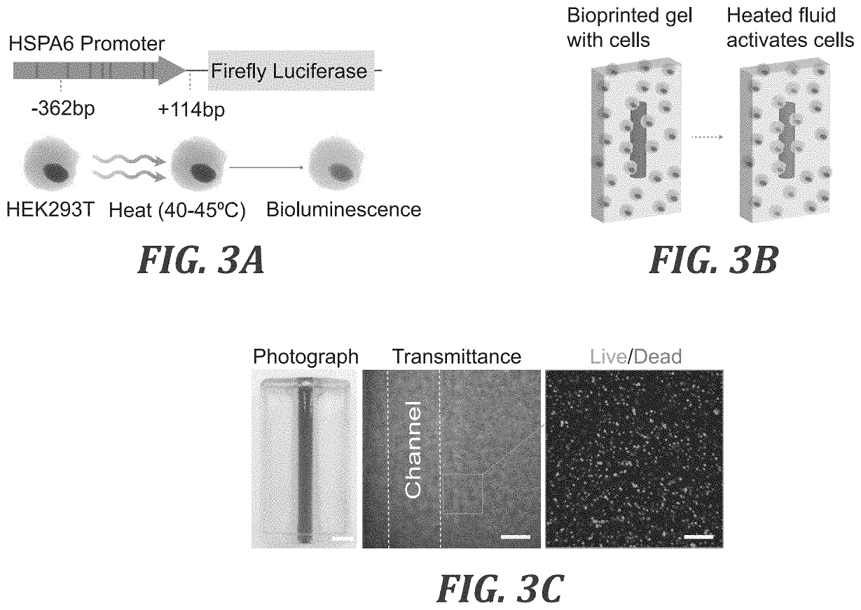

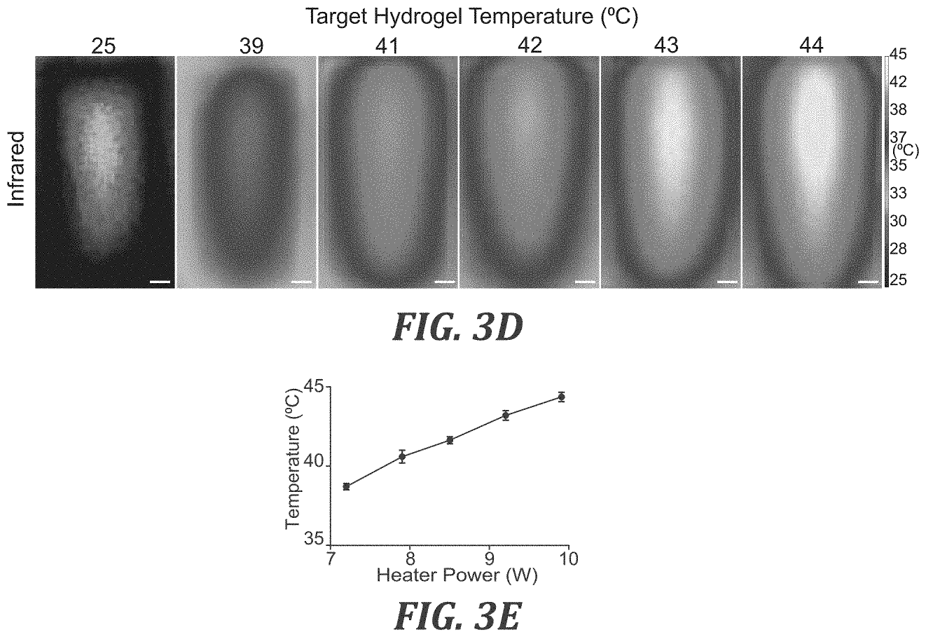

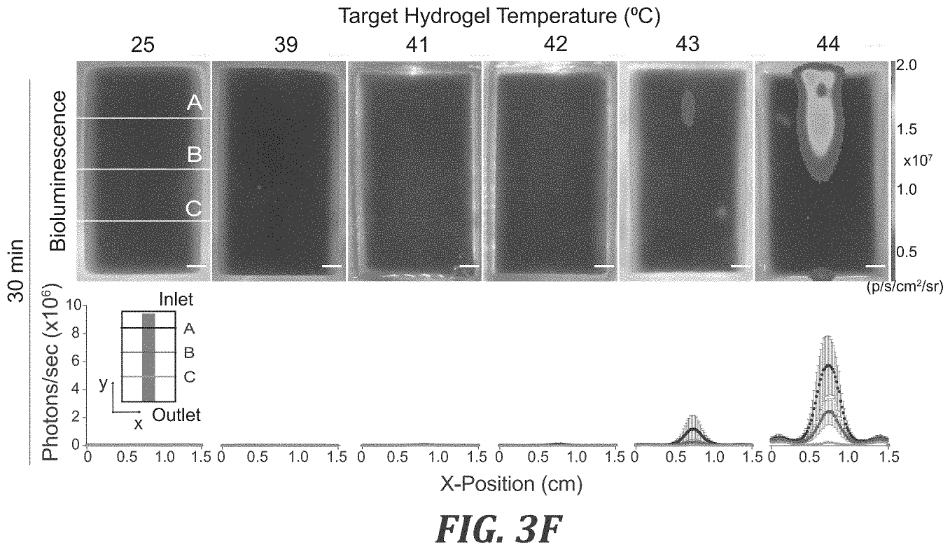

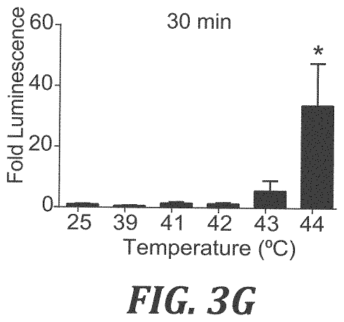

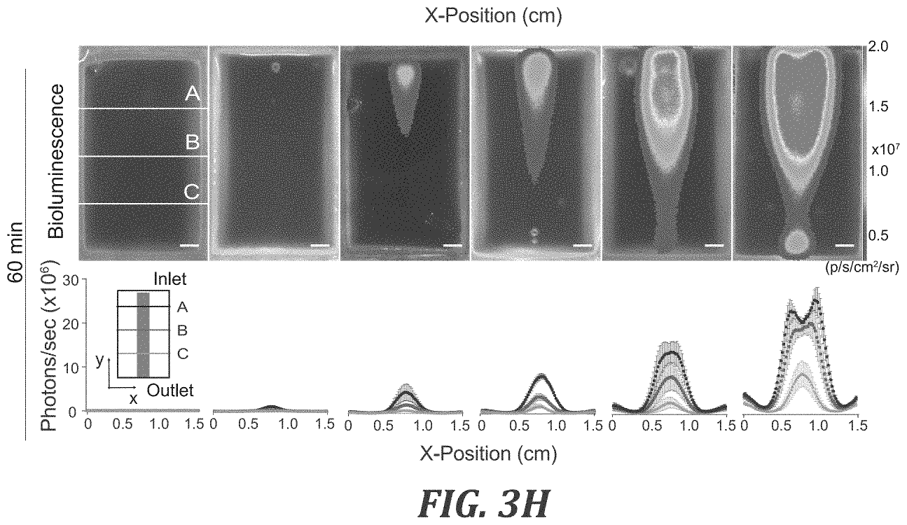

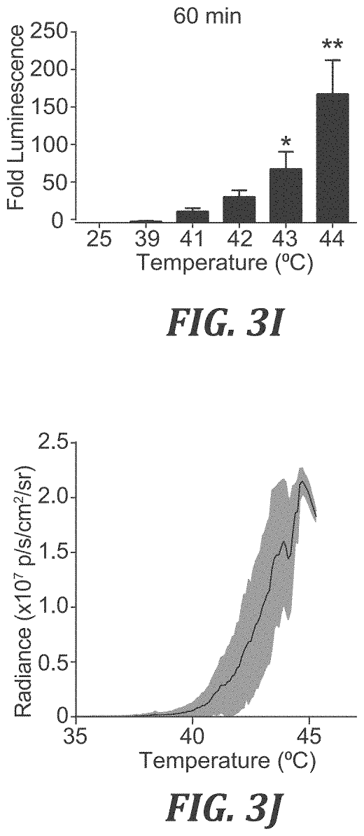

Exemplary genetically engineer heat-inducible cells that activate gene expression upon exposure to mild hyperthermia were produced as follows. To do this, a temperature-responsive gene switch based on the human heat shock protein 6A promoter (HSPA6) was implemented, which exhibits a low level of basal activity and a high degree of upregulation in response to mild heating. This promoter activates heat-regulated transcription through consensus pentanucleotide sequences called heat shock elements, which are binding sites for heat shock transcription factors. HEK293T cells were transduced with a lentiviral construct in which a 476 bp region of the HSPA6 promoter containing eight canonical heat shock elements was placed upstream of a Firefly luciferase reporter gene (fLuc; FIG. 3A). Initial characterization of temperature-sensitive promoter activity in engineered cells in 2D tissue culture demonstrated a temperature-dose dependent upregulation of luciferase activity in the range of mild hyperthermia. Statistically significant upregulation was observed in heated cells compared to non-heated controls after hyperthermia for 30 minutes at 45° C. or 60 minutes from 43-45° C., while peak bioluminescence occurred after 60 minutes at 44° C. (292±26-fold increase in bioluminescence relative to 37° C. controls). Bioluminescent signal was first detected eight hours after heat shock, peaked at 16 hours (110±30-fold increase), and fell back to baseline by two days. Administration of a second heat shock stimulus three days later re-induced bioluminescent signal. Thus, gene activation with this promoter system is transient but can be re-activated with pulsing.

It was observed that the highest heat exposure (45° C. for 60 min) led to a tradeoff between bioluminescence and cell integrity, as indicated by reduced cell metabolic activity and substrate detachment. Without wishing to be bound by theory, these findings suggested that fine control of heat would be needed for thermofluidics to be useful in cellularized applications. The inventors therefore rigorously characterized the effect of heating on HEK293T cells embedded in the exemplary hydrogel formulation used for the thermofluidic studies described herein. Similar to 2D studies, cell viability fell significantly only after exposure to the highest temperature used, 45° C. Taken together, these studies demonstrate engineering of human cells with a heat-sensitive gene switch and identification of a tight window of thermal exposure parameters that both differentially upregulate gene bioluminescence and maintain cell integrity.

Thermofluidic Activation of Gene Expression in Artificial Tissues

The inventors sought to determine whether thermofluidic heating could be used to induce gene expression in heat-inducible cells encased within 3D artificial tissues (FIG. 3B). To do this, heat-inducible cells were encapsulated in the bulk of bioprinted constructs that contained a single perfusable channel (FIGS. 3B and 3C). Since tissue constructs were printed from biocompatible materials without ultraviolet light crosslinking, most cells remained viable upon encapsulation (FIG. 3C). To determine whether these exemplary heat-inducible cells could be activated using thermofluidics, the channels were perfused at 0.5 mL min⁻¹ using thermal exposure parameters identified in 2D culture (FIGS. 3D and 3E). Similar to 2D, the observed thermal dose-dependent luciferase upregulation (FIGS. 3F-3J) was statistically significant after 30 min of heating to a target hydrogel temperature of 44° C., or after 60 min of heating to temperatures of 43° C. and 44° C. by whole-gel bioluminescent output (71±22 fold and 169±44 fold increase relative to controls, respectively; FIGS. 3H and 3I). To more finely characterize how bioluminescent intensity correlates with temperature, infrared and bioluminescence images were overlaid to map individual pixels and generate temperature-bioluminescence response curves. The shape of temperature-response curves appeared similar in shape across various target temperatures (FIG. 3J, all data overlaid). Similar to whole-gel analyses, greater target temperatures generated the most robust activation (FIG. 3J). In initial studies, it was noted that leakage at the hydrogel inlet or outlet could activate cells. Subsequent improvements to fluidic connectivity with a custom-printed perfusion apparatus led to higher precision thermal patterning. Finally, multi-perspective imaging and bioluminescence quantification of single-channel perfused hydrogels from both ‘top-down’ and ‘cross-sectional’ perspectives demonstrated that reporter gene activation had a three-dimensional radial gradient topology around each channel. Taken together, these results illustrate that thermofluidics can be used to activate varying levels of gene expression in 3D artificial tissues.

Heat Exchangers Facilitate Spatial and Dynamic Control of Gene Expression Patterning

Spatial patterns of gene expression within native tissues vary widely in magnitude, scale, and spatial complexity. While variation in magnitude was achieved in the single channel studies, the expression profile geometry across the hydrogel remained similar at various perfusion temperatures. This raised the question of how to design heat delivery schemes that enable more spatially complex expression patterns across the hydrogel. The thermal characterization (FIG. 2) revealed flow rate as one parameter that could be employed but changing flow rate alone imparted only subtle differences to the spatial thermal profile (FIGS. 2D-F). To identify a more perturbative and user-defined means of affecting heat distribution across the hydrogel the inventors turned to industrial heat transfer applications, in which heat exchangers are optimized to transfer heat between fluids by controlling parameters such as channel placement and flow pattern.

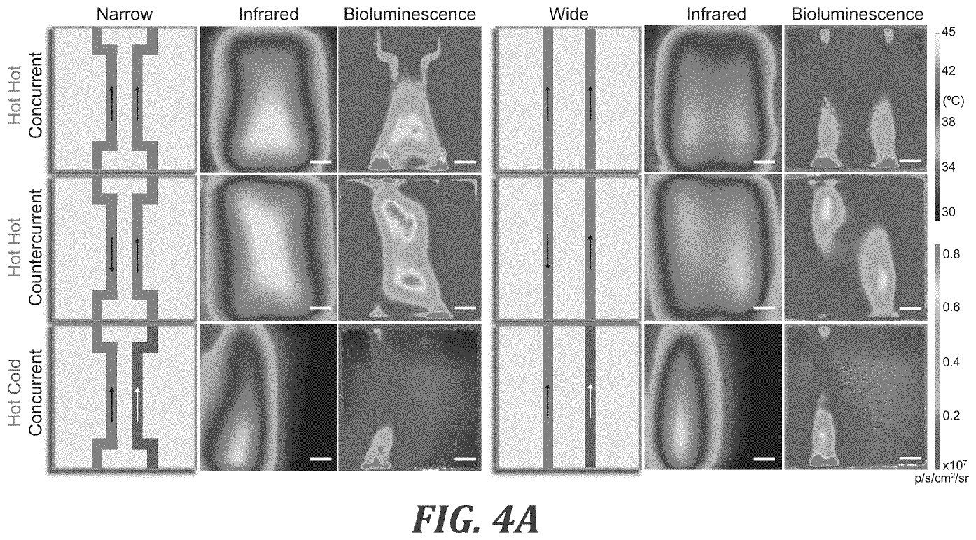

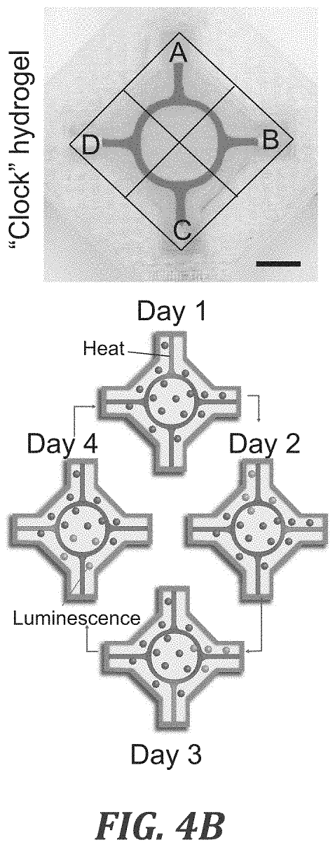

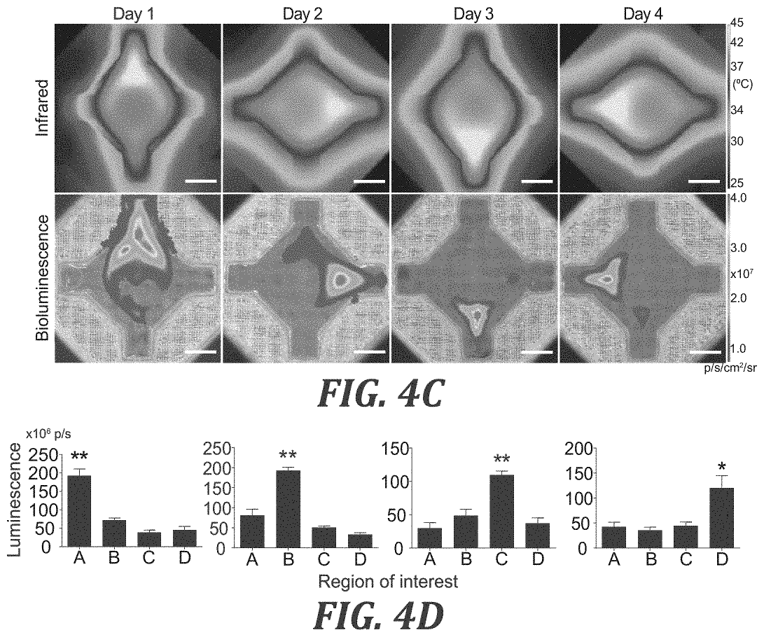

A double pipe heat exchanger design was mimicked within cellularized hydrogels by printing two channels at varying distances from one another (FIG. 4A, narrow vs. wide). The hydrogels were then perfused under different conditions for flow direction (concurrent vs. countercurrent) and fluid temperature (hot, 44° C. vs. cold, 25° C.). Similar to the single-channel characterization, double channel tissues showed close matching between thermal and bioluminescence profiles (FIG. 4A). Concurrent flow in narrow spaced channels created elongated spatial plateaus of heat and bioluminescence between the channels. Conversely, widely spaced hot channels generated mirror image thermal and bioluminescent profiles, with distinct spatial separation between channels. Countercurrent flow patterns generated parallelogrammic thermal and bioluminescent profiles in both channel spacings. Substituting a hot channel for a cold channel attenuated bioluminescence in a manner that depended on channel spacing (FIG. 4A). Computational models of a similar bifurcating channel geometry further demonstrated how simple changes to parameters such as channel spacing can alter spatial thermal profile.

As biological gene expression patterns are transient and fluctuating, it was tested whether thermofluidics could dynamically localize regions of gene expression over time. To do this, clock-inspired constructs were printed, in which four separate inlets converged on a circular channel (FIG. 4B, top). We then perfused heated fluid through each inlet over four consecutive days (FIG. 4B, bottom) and imaged tissues for bioluminescence. Bioluminescent images demonstrated statistically significant luciferase upregulation for regions surrounding heated inlets compared to non-heated inlet regions on all four days (FIGS. 4C and 4D.) Together, these results illustrate that by exploiting heat transfer design principles, thermofluidics enables user-defined spatial and dynamic patterning of mesoscale gene expression patterns in 3D artificial tissues

Gene Patterning is Maintained after In Vivo Engraftment

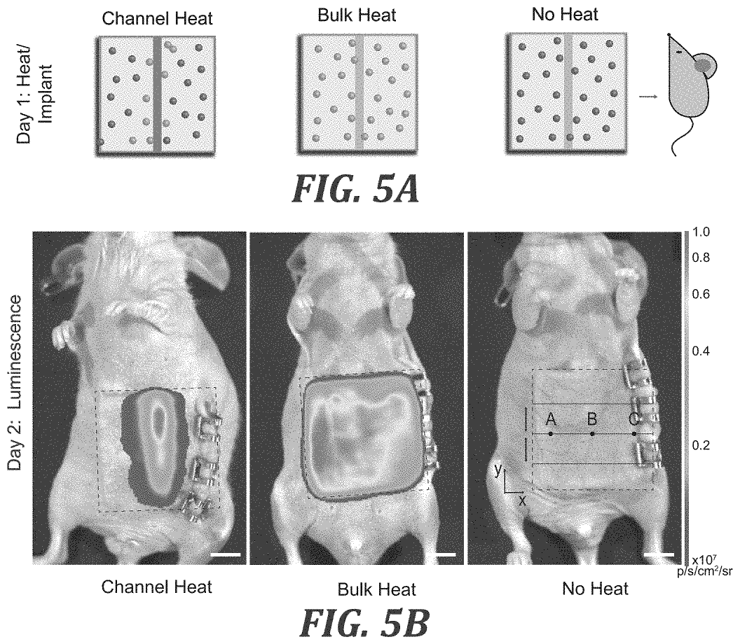

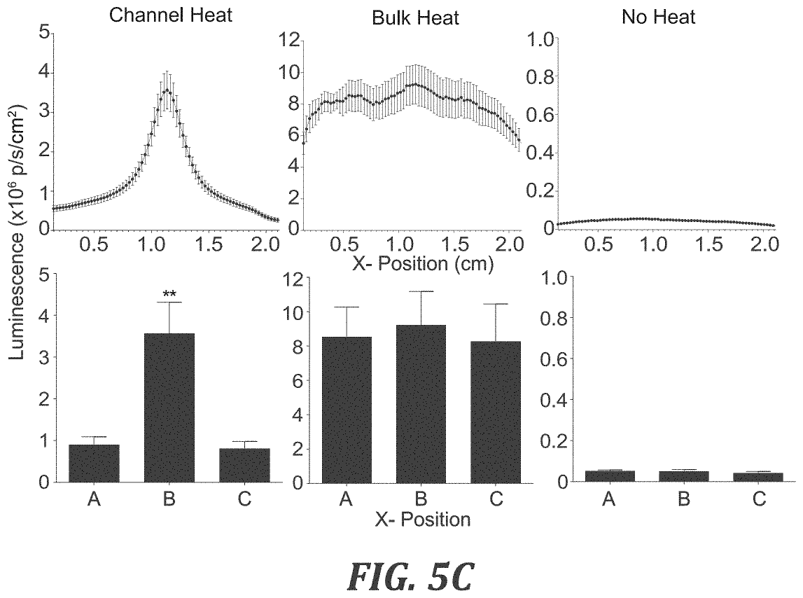

To test whether gene patterning could be maintained after engraftment of artificial tissues in vivo, tissues were stimulated with HEAT and implanted into athymic mice. All tissues contained HEK293T cells expressing fLuc under the control of the heat-inducible HSPA6 promoter. All tissue constructs contained a single channel, and were stimulated in one of three ways: 1) thermofluidic perfusion at 44° C. for 60 min, 2) bulk heating in a cell culture incubator at 44° C. for 60 min, or 3) bulk exposure in a cell culture incubator to 37° C. Tissues were implanted into mice immediately after heating and bioluminescence imaging was performed 24 hours later. It was found that thermofluidic spatial control of gene expression was maintained after in vivo tissue engraftment (FIG. 5A).

Spatial Control of Wnt/β-Catenin Signaling Pathway

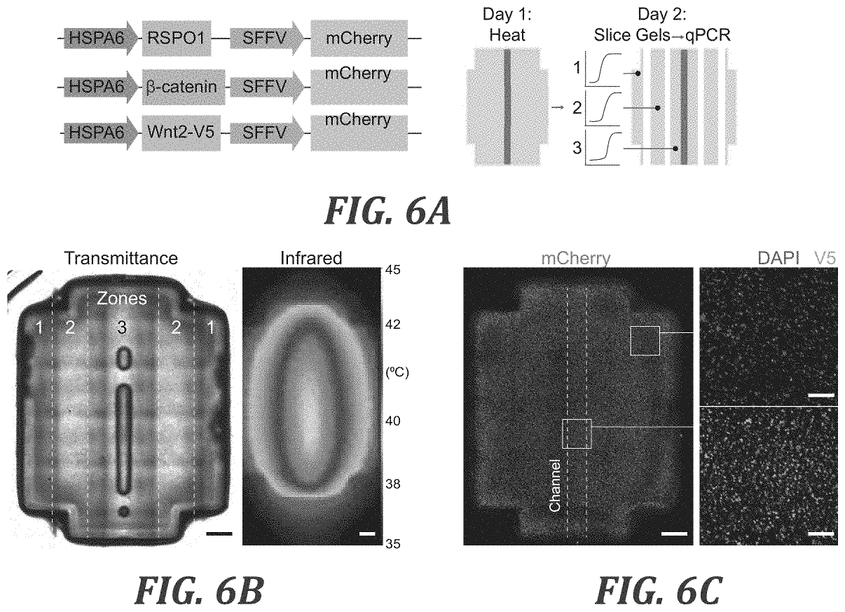

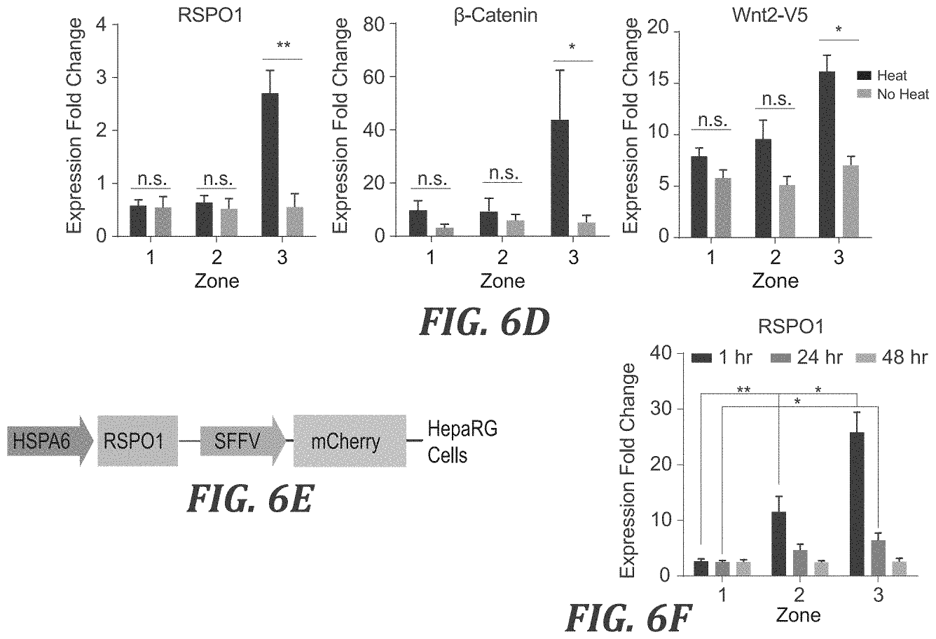

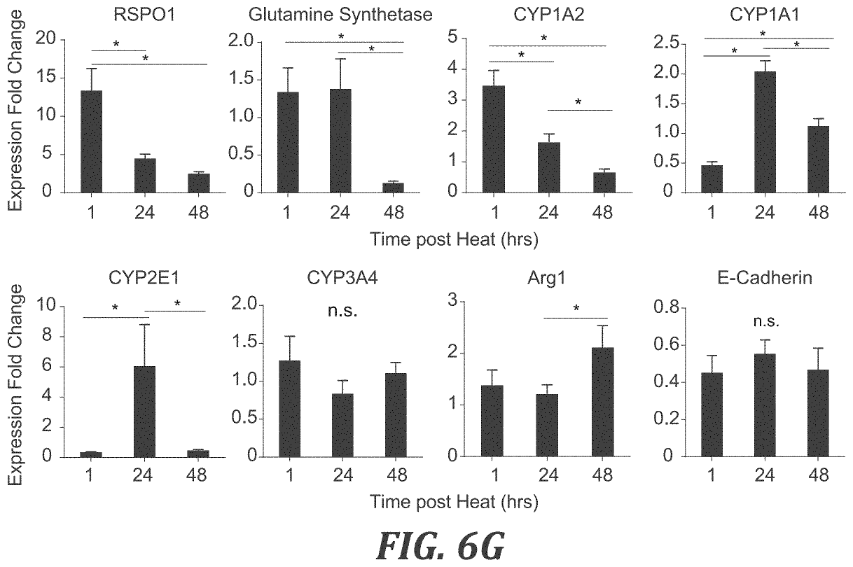

This example demonstrates the modularity of the tissues and methods disclosed herein for spatially regulating expression of the Wnt/β-catenin signaling pathway, which directs diverse aspects of embryonic development, tissue homeostasis, regeneration, and disease. Heat-inducible constructs were engineered to drive expression of three genes in the Wnt/β-catenin signaling pathway: 1) R-spondin-1 (RSPO1), a potent positive regulator of Wnt/β-catenin signaling, 2) β-catenin, a critical transcriptional co-regulator that translates to the nucleus upon canonical Wnt signaling, and 3) Wnt-2, a ligand that binds to membrane-bound receptors to activate the Wnt/β-catenin signaling pathway. The Wnt-2 gene was also tagged with V5. Lentiviral constructs were engineered in which RSPO1, β-catenin, or Wnt2-V5 is driven by the heat-inducible HSPA6 promoter and mCherry is driven by a constitutive promoter (spleen focus-forming virus, SFFV; FIG. 6A). RT-qPCR analysis of each engineered cell line for mCherry expression relative to GAPDH expression suggested lentiviral integration. The artificial tissues containing heat-inducible β-catenin, RSPO1, or Wnt2 HEK293T cells and a single fluidic channel (FIG. 6B) were then printed. Constructs were heated fluidically and then sliced into longitudinal zones (FIG. 6A, B) to analyze expression of the Wnt family gene expression by RT-qPCR. Representative artificial tissues contained mCherry positive cells across the tissue (FIG. 6C). Immunostaining for the V5 tag fused to Wnt2 appeared higher near the heated channel compared to the gel periphery (FIG. 6C). R-spondin-1, β-catenin, or Wnt2 expression was highest in the zone surrounding the heated channel FIG. 6D). These results show that HEAT can be leveraged to activate expression of various family members of the Wnt/β-catenin signaling pathway.

Thermofluidic Activation of R-Spondin-1 Drives Expression of Key Metabolic Liver Enzymes

It was reasoned that the ability to activate expression of Wnt/β-catenin signaling pathway members could be useful for the emerging human “organ-on-a-chip” field, by affecting functional cellular phenotypes in vitro. To test this, the inventors turned to the liver, which performs hundreds of metabolic functions essential for life, including central roles in drug metabolism. To carry out these functions, hepatocytes divide the labor, with hepatocytes in different spatial locations performing different functions, a phenomenon called liver zonation. Recent studies have shown that liver zonation is regulated at the molecular level by Wnt/β-catenin signaling, with higher Wnt activity associated with a pericentral vein phenotype and lower Wnt activity characteristic of a periportal phenotype. However, the extent to which different members of this pathway affect human zonated hepatic phenotypes remains unclear. A better understanding of this process would accelerate development of zonated human liver models for hepatotoxicity and drug metabolism studies.

Without wishing to be bound by theory, the inventors hypothesized that thermofluidic activation of R-spondin-1 in human hepatic cells would be sufficient to activate zonated hepatic gene expression profiles, as ectopic expression of RSPO1 in mouse liver has recently been shown to induce a pericentral zonation phenotype in vivo. To test this hypothesis, human HepaRG cells, an immortalized human hepatic cell line that retains characteristics of primary human hepatocytes, was transduced with the lentiviral construct disclosed herein in which HSPA6 drives RSPO1 and SFFV drives mCherry (FIG. 6E). Transduced human hepatic cells were then printed in artificial tissues with a single fluidic channel, to mimic central lobular placement of the central vein (FIG. 6E). Constructs were heated fluidically and then sliced into zones (FIG. 6A) and gene expression was measured by RT-qPCR (FIG. 6F). Fold upregulation values were normalized to identically fabricated control artificial tissues maintained at 37° C. It was found that RSPO1 expression increased in a dose-dependent and spatially defined manner, with expression in Zone 3 nearest the channel (“central vein”) 10-fold higher than in Zone 1 by one-hour post heating. RSPO1 expression was transient, falling with each day after heating, similar to the above described luciferase studies. Importantly, thermofluidic activation of RSPO1 induced expression of key pericentral marker genes, including glutamine synthetase, an enzyme involved in nitrogen metabolism, and the cytochrome P450 (CYP) drug-metabolizing enzymes CYP1A2, CYP1A1, and CYP2E1 relative to control tissues that were not heated, though with varied timing and without spatial localization in this study (FIG. 6G). Expression of pericentral drug-metabolizing enzyme CYP3A4 was not induced with heating, consistent with other studies in which adding Wnt3a ligand to primary human hepatocyte cultures did not alter CYP3A4 expression. Periportal marker E-Cadherin was not induced, but periportal/midzonal gene Arg-1 increased at 48 hours, especially in the Zone 2 midzonal region. Taken together, these examples contribute a fundamental understanding of how various liver zonation genes are induced by RSPO1 activation in human hepatic cells

Discussion

The examples provided herein demonstrate that thermal patterning via bioprinted fluidics can directly pattern gene expression in 3D artificial tissues. A key advantage of the HEAT method is that it leverages the recent explosion in accessible additive manufacturing tools by using bioprinting methods that are readily available to the broader community. Furthermore, the entire patterned network is stimulated nearly simultaneously (as opposed to sequentially by time-intensive rastering), and this parallel stimulation can be sustained for exposure times required to trigger gene expression. Together, the sheer rapidity and highly parallel nature of this process enable spatial and dynamic genetic patterning at length scales and depths not previously possible in 3D artificial tissues.

Previously reported methods to elicit cellular signaling in artificial tissues have focused on tethering extracellular cues to hydrogels. Innovations in stimuli-responsive or ‘smart’ biomaterials enabled activation of such chemistries by exogenous physical stimuli, such as light, to control the spatial position and timing of extracellular cues. Although useful, such materials-focused methods are unlikely to provide complete control even in fully defined starting environments because cells rapidly remodel their microenvironments. Moreover, such technologies offer an imprecise means to control downstream transcription because many, often unknown, intermediary steps modify intracellular signal transduction prior to gene activation. The thermofluidic methods and tissues of the disclosure provide a complementary new technology to such methods that target extracellular signals by facilitating spatiotemporal control at the intracellular genetic level.

The data presented herein here reveal the potential power of HEAT for gene patterning, the exemplary tissues, methods, and systems presented herein. Even though it was found that channels up to 30 mm long (but no longer) could achieve hyperthermic temperature ranges along the entire channel length and the effect of heat-mediated stimulation on gene expression was transient, these limits can be overcome through a variety of design modifications. For example, the thermal conductivity of the hydrogel of the tissues disclosed herein or perfusate could be increased by materials engineering to extend patterning area or length, such as by crosslinking metal nanoparticles into the polymer backbone as has been done before for other applications. To achieve different activation temperatures or dynamics, further genetic engineering of the heat shock promoter or other heat-activatable gene switches could be employed.

To fully realize the vision of precision-controlled 3D artificial tissues, a diverse toolkit of orthogonal physical delivery and molecular remote-control agents can be used. Thermofluidics can be coupled with other tissue engineering strategies that program extracellular or intracellular signal presentation, cell patterning, or tissue curvature. Thermofluidics can also be used orthogonally with other remote-control agents, such as those leveraging small molecule, ultrasound, radio wave, magnetic, or light-based activation. Coupled with rapid advances in gene editing, synthetic morphogenesis, and stem cell technology, thermofluidics can be useful for spatially and temporally activating genes across tissues to drive cell proliferation, fate or assembly decisions. While the utility for activating Wnt/β-catenin signaling pathway genes was demonstrate here, this approach can be adapted to activate any gene of interest. One application of this approach has been demonstrated herein, by driving human hepatic cells towards a more pericentral liver phenotype in 3D artificial tissues. In doing so, fundamental insights into how activation of Wnt agonist RSPO1 regulates expression of various metabolic zonation genes was gained. These findings have important implications for developing both organ-on-chip systems for pharmacology and hepatotoxicity, as well as artificial tissues for human therapy. By blurring the interface between the advanced fabrication and biological realms, thermofluidics thus creates a new avenue for bioactive tissues with applications in both basic and translational biomedicine.

While illustrative embodiments have been illustrated and described, it will be appreciated that various changes can be made therein without departing from the spirit and scope of the invention.

The embodiments of the invention in which an exclusive property or privilege is claimed are defined as follows:

1. A biocompatible heat exchanger, comprising:
a three-dimensional thermally conductive hydrogel substrate comprising at least one fluid-perfusable channel, wherein the at least one fluid-perfusable channel comprises an inlet, an outlet, and a flow region in fluid communication with the inlet and the outlet and configured to contain a flow of a fluidic medium; and
one or more heat-inducible elements configured to generate one or more biological signals when heated above or cooled below a threshold temperature.

2. The biocompatible heat exchanger of claim 1, wherein the one or more heat-inducible elements are cells genetically modified to comprise a heat inducible promoter or enhancer operatively linked to a gene of interest.

3. The biocompatible heat exchanger of claim 1, wherein the one or more heat-inducible elements are nanoparticles comprising one or more bioactive moieties or liposomes encapsulating one or more bioactive moieties.

4. The biocompatible heat exchanger of claim 3, wherein the biocompatible heat exchanger comprises a plurality of cells.

5. The biocompatible heat exchanger of claim 1, wherein the flow region is linear.

6. The biocompatible heat exchanger of claim 1, wherein the flow region is non-linear.

7. The biocompatible heat exchanger of claim 1, wherein the flow region comprises at least one first multifurcation downstream from the inlet, at least one first recombination upstream from outlet, and a plurality of second channels fluidly connecting the first multifurcation to the first recombination.

8. The biocompatible heat exchanger of claim 7, wherein the one or more of the second channels comprises at least one second multifurcation downstream from the first multifurcation, at least one second recombination upstream from the first recombination, and a plurality of third channels fluidly connecting the second multifurcation to the second recombination.

9. The biocompatible heat exchanger of claim 7, wherein the plurality of second channels are interconnected into a grid architecture, a spherical architecture, a cubed architecture, or a rectangular cuboid architecture.

10. The biocompatible heat exchanger of claim 1, comprising a plurality of fluid-perfusable channels, wherein at least two of the plurality of fluid-perfusable channels are not in fluid communication.

11. The biocompatible heat exchanger of claim 1, wherein the at least one fluid-perfusable channel comprises a valve configured to controllably regulate flow of fluid in the channel.

12. The biocompatible heat exchanger of claim 1, wherein the at least one fluid-perfusable channel has a variable diameter along its length.

13. The biocompatible heat exchanger of claim 2, wherein the one or more heat inducible promoters or enhancers is selected from the group consisting of a heat shock protein promoter, an RNA thermometer promoter, a transient receptor potential cation channel (TRPV) promoter, phage lambda pL promoter, phage lambda pR promoter, HSPB, HSP16F, HSPA1A, HSPA1B, HSPA2, and Gal80-intein.

14. A system, comprising the biocompatible heat exchanger of claim 1 and a pump configured to controllably perfuse fluidic medium into at least one inlet of the at least one fluid-perfusable channel.

15. The system of claim 14, further comprising a controllable heating element configured to control the temperature of the fluidic medium.

16. The system of claim 14, further comprising a detector element configured to measure heat in the artificial tissue.

17. The system of claim 18, wherein the detector element comprises an infrared camera, a thermocouple, a thermistor, a thermochromic ink, thermochromic dyes, or a combination thereof.

18. An artificial tissue comprising the biocompatible heat exchanger of claim 1.

19. An artificial tissue configured for thermofluidic control of gene expression, comprising:
a biocompatible three-dimensional hydrogel substrate comprising at least one fluid-perfusable channel, wherein the at least one fluid-perfusable channel comprises an inlet port, an outlet port, and a flow region in fluid communication with the inlet and the outlet ports and configured to contain a flow of a fluidic medium; and
a plurality of cells genetically modified to comprise a heat inducible promoter or enhancer operatively linked to a gene of interest.

20. A method of controlling gene expression in a three-dimensional space, comprising:
providing a plurality of genetically modified cells comprising a heat inducible promoter or enhancer operatively linked to a gene of interest in a three dimensional hydrogel substrate, wherein the three dimensional hydrogel substrate comprises at least one fluid-perfusable channel, wherein the at least one fluid-perfusable channel comprises an inlet port, an outlet port, and a flow region in fluid communication with the inlet and the outlet ports and configured to contain a flow of a fluidic medium; and
perfusing a sufficient volume of a heated fluid into the at least one fluid-perfusable channel through the at least one inlet to activate expression of the gene of interest.
